(19) United States
(12) Patent Application Publication
(10) Pub. No.: US 2008/0181919 A1
(43) Pub. Date: Jul. 31, 2008

(54) SHIUNKO NANOMICELL FOR SKIN TREATMENT AND PREPARATION METHOD FOR THE SAME

(75) Inventors: Chih-Hao Chang, Kaohsiung County (TW); Tong-Rong Tsai, Kaohsiung City (TW); Wen-Lung Hu, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 11/845,171

(22) Filed: Aug. 27, 2007

(30) Foreign Application Priority Data
Jan. 31, 2007 (TW) 96103618

(51) Int. Cl. A61K 36/232; A61K 36/23; A61K 9/00; A61P 17/02

(52) U.S. Cl. 424/400; 424/773

(57) ABSTRACT

The present invention provides a nanomicell for a skin, and the nanomicell includes an oil substance, an extract of a Angelica Radix, an extract of a Lithospermum Radix, and a phospholipid layer. The extract of a Angelica Radix is formed by extracting the Angelica Radix with the oil substance, and the extract of a Lithospermum Radix is formed by extracting the Lithospermum Radix with the oil substance. The extract of the Angelica Radix and the extract of the Lithospermum Radix are packaged within the phospholipid layer to form a plurality of micells having a diameter of nano-level.

### FIELD OF THE INVENTION

This invention relates to an gel for skin treatment and preparation method for the same, and more particularly to an Shiunko nanomicell for skin treatment and preparation method for the same.

### BACKGROUND OF THE INVENTION

Shiunko is a Chinese traditional ointment used for treating burns and scalds, and mainly consists of Lithospermum Radix, Angelica Radix, Sesame oil, and Wax. The effective components of Lithospermum Radix includes shikonin and the derivatives thereof, which have been proved having the functions of granulation tissue formation improving, anti-inflammatory, fasting the wound healing, anti-bacteria, and anti-tumor. Shiunko also has the advantages of cheap price and easy preparation. However, Shiunko also has the deficiencies of being easy denaturalized under room temperature, thick sesame smell, oily sensation, hard to cleaning so that it is inconvenient in clinical use.

Silver sulfadiazine 1% cream is the major agent used to treat burns and scalds in western medicine from 1968, and silver sulfadiazine has the function of inducing the cell wall deformation of the bacteria, damaging the cell membrane so that it can kill the bacteria. However, the silver granules will deposit in skin dermis to cause Argyrai, and in monolayer culture, the silver ions and the sulfadiazine are even found having a high toxicity in keratinocytes and fibroblasts, which is harmful to wound healing. Further, the toxic dose and the sterilization dose of the silver ions are similar, and hence the normal cells will be damaged when the bacteria are killed. In addition, sulfadiazine will also inhibit the bone marrow function and induce allergy.

The technology of nanomicell is developed recently, which is a concentrated solution of phospholipids and certain solvents such as a glycerin or a Propylene Glycol. The nanomicell can dissolve the oil and substances that are difficult to be dissolved in water under room temperature (25° C.-60° C.). The concentrated solution will form numerous liposomes if diluted with water, and hence the nanomicell means a transparent and concentrated solution that includes the lipid-soluble drugs packaged with phospholipids and water to form numerous micells. The nanomicell has advantages of well stability, high hydrophile, safety in physiology, no irritation, no synthesized detergent and preservative using, and even can improve the absorption of active components and bio-availability. The nano-sized micells can pass through the cell membrane by cell fusion, endocytosis or phagocytosis, and hence are suitable as the excipient in cosmetic, pharmaceutical or biomedical research. Further, the micell have a diameter less than 100 nanometers can be absorbed by skin, which is an ideal transdermal drug delivery method that is efficient and not invasive.

U.S. Pat. No. 6,468,553 and Taiwan Pat. No. I226,838 have made efforts on the oil substrate of the traditional Shiunko, but the deficiencies of oily sensation and hard to cleaning still exist. Further, the improvement in prior art maybe prolong the storage time under room temperature, but has no amelioration in transdermal ability.

In order to overcome the drawbacks in the prior art, an Shiunko nanomicell for skin treatment is provided. The particular design in the present invention not only solves the problems described above, but also is easy to be implemented. Thus, the invention has the utility for the industry.

### SUMMARY OF THE INVENTION

It is an aspect of the present invention to provide a nanomicell for a skin, and the nanomicell includes an oil substance, an extract of a Angelica Radix, an extract of a Lithospermum Radix, and a phospholipid layer. The extract of a Angelica Radix is formed by extracting the Angelica Radix with the oil substance, and the extract of a Lithospermum Radix is formed by extracting the Lithospermum Radix with the oil substance. The extract of the Angelica Radix and the extract of the Lithospermum Radix are packaged within the phospholipid layer to form a plurality of micells having a diameter of nano-level.

Preferably, the oil substance is one selected from a group consisting of a sesame oil, a mineral oil, and an olive oil.

Preferably, the micell further includes an agueous layer outside the phospholipids layer.

Preferably, the micell has a diameter lesser than 100 nanometers.

Preferably, the nanomicell is used for a treatment for one selected from a group consisting of wound injury, burn injury, dry skin, fissure, frostbite, ulcer, and proliferate skin diseases.

It is another aspect of the present invention to provide a nanomicell gel for a skin treatment. The nanomicell gel includes an excipient that has a plurality of micells distributed therein. Further, each of the plurality of micells includes an oil extract of Angelica Radix, an oil extract of Lithospermum Radix, and a phospholipid layer. The phospholipid layer wraps therewithin the oil extract of Angelica Radix and the oil extract of Lithospermum Radix.

Preferably, the phospholipids layer includes a glycerin and a phospholipids.

Preferably, each of the plurality of micells further includes a aqueous layer outside the phospholipid layer.

Preferably, the excipient is selected from one of a gel and a hydrophilic ointment.

Preferably, each of the plurality of micells has a diameter lesser than 100 nanometers.

Preferably, the nanomicell gel is used for a treatment for one selected from a group consisting of wound injury, burn injury, dry skin, fissure, frostbite, ulcer, and proliferate skin diseases.

It is further another aspect of the present invention to provide a method of forming a nanomicell for a skin. The method includes steps of (a) extracting an Angelica Radix and a Lithospermum Radix with an oil substance to obtain an extract; (b) adding a phospholipid and a water into the extract; (c) stirring the extract in a relatively high speed and relatively high pressure condition to homogenize the extract; and (d) filtrating the extract.

Preferably, the method further includes a step of soaking the Angelica Radix in the oil substance for 24 hours before the step of extracting an Angelica Radix and a Lithospermum Radix.

Preferably, the method further includes a step of heating the oil substance to a temperature between 130° C. and 140° C. to exact the Angelica Radix and the Lithospermum Radix.

Preferably, the step (d) is carried out by a filter having a pore size below 0.1 μm.

Preferably, the oil substance is one selected from a group consisted of a sesame oil, a mineral oil, and an olive oil.

Preferably, the method further includes a step of add a excipient to the extract after the step of filtrating the extract.

Preferably, the excipient is one selected from a gel and a hydrophilic ointment.

The above aspects and advantages of the present invention will become more readily apparent to those ordinarily skilled in the art after reviewing the following detailed description and accompanying drawings, in which:

### BRIEF DESCRIPTION OF THE DRAWINGS

FIG. 1 is a polygon illustrating the variation in particle size of the different nanomicells after different storage times according to the embodiment of the present invention;

FIG. 2(A) is a diagram showing the bacterial culture results of Pseudomonas aeruginosa after 0, 8, and 24 hours of culture with no drugs (control group)(a), silver sulfadiazine 1% cream(b), or traditional Shiunko(c) respectively according to the embodiment of the present invention;

FIG. 2(B) is a diagram showing the bacterial culture results of Pseudomonas aeruginosa after 0, 8, and 24 hours of culture with SSN(d), OSN(e) or MSN(f) respectively according to the embodiment of the present invention;

FIG. 3 is a diagram showing the wounds of the experimental rabbits at the time of 31 days after the burn injury and successive treatment of no drugs(a), silver sulfadiazine 1% cream(b), traditional Shiunko(c), SSN(d), OSN(e), and MSN(f) respectively according to the embodiment of the present invention, (and the circle area points out the original scald wound);

FIG. 4 is a diagram showing the wounds of the experimental rabbits at the time of 37 days after the burn injury and successive treatment of no drugs(a), silver sulfadiazine 1% cream(b), traditional Shiunko(c), SSN(d), OSN(e), and MSN(f) respectively according to the embodiment of the present invention (the circle area means the original scald wound); and

FIGS. 5(A)-(F) are diagrams showing wound tissue sections in LM 20× after stained with Hematoxylin & Eosin at the time of 20 days after the burn injury and successive treatment of no drugs(a), silver sulfadiazine 1% cream(b), traditional Shiunko(c), SSN(d), OSN(e), and MSN(f) respectively according to the embodiment of the present invention.

### DETAILED DESCRIPTION OF THE PREFERRED EMBODIMENT

The invention is described more specifically with reference to the following embodiments. It is to be noted that the following descriptions of preferred embodiments of this invention are presented herein for the purpose of illustration and description only; it is not intended to be exhaustive or to be limited to the precise form disclosed.

The Lithospermum Radix used in the following experiments is Arnebia euchroma, and the Angelica Radix is the dry roots of Angelica sinensis (Oliv.) Diels. The preparing method of different Shiunko Nanomicells will be introduced first, and then the creativeness of the Shiunko Nanomicells will be showed by different function testing experiments.

### The Preparing Methods of Shiunko Oils Containing Different Oil Substance

The materials in S group include: Lithospermum Radix 90 gm, Angelica Radix 90 gm, and sesame oil 300 gm. The materials in group 0 includes: Lithospermum Radix 90 gm, Angelica Radix 90 gm, and olive oil 300 gm, and the materials in group M includes: Lithospermum Radix 90 gm, Angelica Radix 90 gm, and mineral oil 300 gm. The preparing method of Shiunkos include steps of: first, purifying the Angelica Radix by eliminating the impurities, and then soaking the Angelica Radix in different oil substances as listed above. After 24 hours, the oil substances containing Angelica Radix are heated to a temperature between 130° C. to 140° C. until Angelica Radix has a scorched surface, and then adding Lithospermum Radix to the oil substances and keeping in the same temperature for 15 minutes. Second, when the oil substances having a red color, heating is stopped, and then Angelica Radix and Lithospermum Radix are removed from the respective oil substances. Finally, the oil substance and the extract of Angelica Radix and Lithospermum Radix therein are filtered with 4 layers of sterile swabs respectively, and then the respective filtrates are stored in different glass bottles.

### The Preparing Methods of Shiunko Nanomicells

The materials include: the Shiuriko oils sampled from the group S, the group O and the group M respectively, and further includes glycerin, phospholipids and pure water. The steps of the preparing methods include: First, adding the phospholipids into the samples S, O, and M respectively, and stirring uniformly to get respective mixtures. Second, the respective mixtures above are added into the glycerin respectively and stirred, and then the pure water is also added into. After high speed stirred in homogenizer (6000 rpm, 3 minutes), the respective mixtures are homogenized in high pressure homogenizer, and filtered with 0.1 g/m filter membrane. Finally, the condensed solutions of a sesame Shiunko nanomicell (group S), olive Shiunko nanomicell (group 0), and mineral Shiunko nanomicell (group M) having a red color are collected respectively.

### The Preparing Methods of Shiunko Nanomicell Gels

The Shiunko nanomicell gels could be made by mixing 100 ml of respective Shiunko nanomicells mentioned above with 100 gm of transparent gel (Lubrajel DV), and then stored in bottles.

### Function Test I: Particle Size Analysis

Drops of sesame Shiuriko nanomicell(SSN), olive Shiunko nanomicell(OSN), and mineral Shiuriko nanomicell(MSN) are sampled respectively, and diluted in 100 ml of pure water to form diluents respectively. After stirring homogeneously, drops of the respective diluents are sampled and analyzed by a particle size analyzer (Beckman Coulter™N5). The results are showed in table 1.

TABLE 1

| | | |
|---|---|---|
| SSN | OSN | MSN |

| | | | |
|---|---|---|---|
| Day 1 | 66.6 ± 25.8 nm | 80.5 ± 25.5 nm | 70.8 ± 27.7 nm |
| Day 20 | 99.0 ± 31.6 nm | 79.1 ± 25.0 nm | 44.7 ± 20.4 nm |
| Day 30 | 78.7 ± 27.5 nm | 91.2 ± 30.5 nm | 75.8 ± 26.7 nm |
| Day 40 | 71.9 ± 22.4 nm | 78.3 ± 23.4 nm | 80.1 ± 24.6 nm |
| Day 50 | 76.2 ± 22.7 nm | 80.7 ± 26.9 nm | 92.9 ± 31.2 nm |
| Day 60 | 73.4 ± 29.4 nm | 90.2 ± 30.9 nm | 66.4 ± 23.2 nm |
| Day 150 | 86.9 ± 40.9 nm | 89.1 ± 28.8 nm | 84.0 ± 24.6 nm |

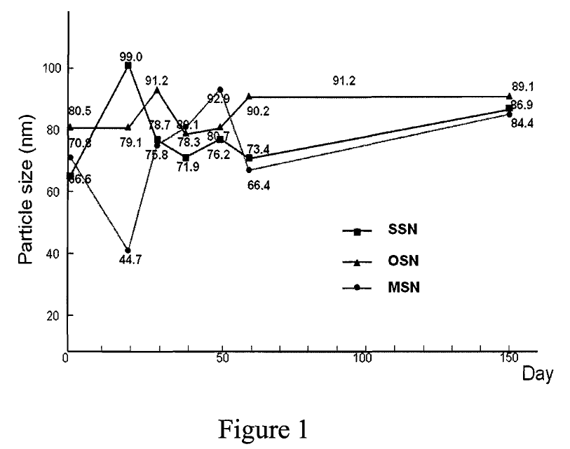

Please also refer to FIG. 1, which is a polygon illustrating the variation in particle size of the different nanomicells after different storage times according to the embodiment of the present invention. As FIG. 1 shows, SSN, OSN and MSN all have a particle size below 100 nm, which are stable even stored under the room temperature for 150 days.

### Function Test II: Anti-Bacteria Test

The bacteria tested in the present invention include Pseudomonas aeruginosa, Escherichia coli, Enterococcus faecalis, Proteus mirabilis and Acinetobacter baumannii, which are usually discovered in wounds of scalds and burns. The bacteria tested in the present invention are showed in table 2.

TABLE 2

| Bacteria | No. |
|---|---|
| Pseudomonas aeruginosa | NCTC 10662 |
| Escherichia coli | ATCC 35218 |
| Enterococcus faecalis | ATCC 29212 |
| Proteus mirabilis | ATCC 7002 |
| Acinetobacter baumannii | ATCC 19606 |

10 μl of respective bacteria fluids of above-mentioned bacteria, of which the total number of bacteria are 10⁴ to 10⁵, are sampled and mixed with 1 ml of Tryptic Soy Broth containing no drugs(a), silver sulfadiazine 1% cream(b), traditional Shiunko(c), SSN, OSN, and MSN respectively, and stirred homogeneously in respective bottles. After 0 minute, 8 hours and 24 hours of culture, 10 μl of culture medium are sampled from the respective bottles, and plated on Horse Blood Agar to subculture in 35° C. for 24 hours. The growing conditions are recorded and the colony forming units are calculated.

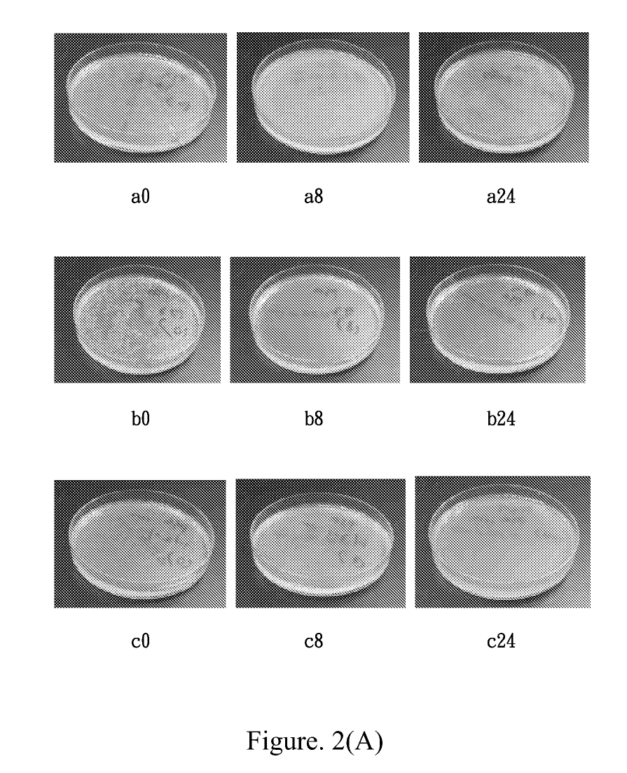

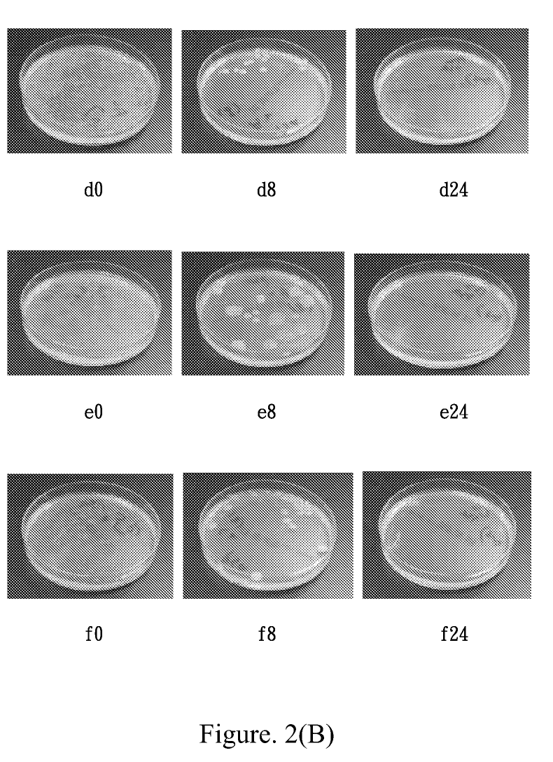

The calculated results of respective bacteria are showed in Table 3 to Table 7. Please also refer to FIGS. 2(A)(B), which are diagrams showing the bacterial culture results of Pseudomonas aeruginosa after 0, 8, and 24 hours of culture with no drugs (control group)(a), silver sulfadiazine 1% cream(b), traditional Shiunko(c), SSN(d), OSN(e) or MSN(f) respectively according to the embodiment of the present invention. Accordingly, the nanomicells provided according to the preferred embodiment of the present invention are actually have the ability in inhibition of Pseudomonas aeruginosa growing, while Pseudomonas aeruginosa is the commonest bacteria existed in wounds of burns and scalds and can easily induce Bacteremia that has a high fatality rate. The detail results are introduced as follows:

(I) Pseudomonas aeruginosa

The calculating results of colony forming units of Pseudomonas aeruginosa are showed in table 3.

TABLE 3
the colony forming units of NCTC 10662 Pseudomonas aeruginosa

| | | silver | | | | |
|---|---|---|---|---|---|---|
| | control | sulfadiazine | traditional | | | |
| | group | 1% cream | Shiunko | SSN | OSN | MSN |
| 0 hours | >10⁵ | >10⁵ | >10⁵ | >10⁵ | >10⁵ | >10⁵ |
| 8 hours | >10⁵ | 0 | >10⁵ | 5 | 10 | 26 |
| 24 hours | >10⁵ | 0 | >10⁵ | 0 | 0 | 0 |

As the table shows, first, in silver sulfadiazine 1% cream treating group, after 0, 8, and 24 hours of culture, the colony forming units are more than 10⁵, 0, and 0 respectively, which shows the silver sulfadiazine 1% cream has a great ability in inhibition of Pseudomonas aeruginosa. Second, in the traditional Shiunko treating group, the colony forming units calculated show that the traditional Shiunko has no ability in inhibition of Pseudomonas aeruginosa. Third, in the SSN treating group, after 0, 8, and 24 hours of culture, the colony forming units are more than 10⁵, 5 and 0 respectively, which shows the SSN has a good ability in inhibition of Pseudomonas aeruginosa. Fourth, in the OSN treating group, after 0, 8, and 24 hours of culture, the colony forming units are more than 10⁵, 10 and 0 respectively, which shows the SSN has a good ability in inhibition of Pseudomonas aeruginosa. Fifth, in the MSN treating group, after 0, 8, and 24 hours of culture, the colony forming units are more than 10⁵, 26 and 0 respectively, which shows the SSN has a good ability in inhibition of Pseudomonas aeruginosa.

(II) Escherichia coli

The calculating results of colony forming units of Escherichia coli are showed in table 4.

TABLE 4
the colony forming units of ATCC 35218 Escherichia coli

| | | silver | | | | |
|---|---|---|---|---|---|---|
| | control | sulfadiazine | traditional | | | |
| | group | 1% cream | Shiunko | SSN | OSN | MSN |
| 0 hours | >10⁵ | >10⁵ | >10⁵ | >10⁵ | >10⁵ | >10⁵ |
| 8 hours | >10⁵ | 0 | >10⁵ | >10⁵ | >10⁵ | >10⁵ |
| 24 hours | >10⁵ | 0 | >10⁵ | >10⁵ | >10⁵ | >10⁵ |

As the table shows, first, the silver sulfadiazine 1% cream has a great ability in inhibition of Pseudomonas aeruginosa. Second, the traditional Shiunko has no ability in inhibition of Escherichia coli. Third, the SSN, OSN, and MSN have no ability in inhibition of Escherichia coli.

(II) Enterococcus faecalis

The calculating results of colony forming units of Enterococcus faecalis are showed in table 5.

TABLE 5
the colony forming units of ATCC 29212 Enterococcus faecalis

| | | silver | | | | |
|---|---|---|---|---|---|---|
| | control | sulfadiazine | traditional | | | |
| | group | 1% cream | Shiunko | SSN | OSN | MSN |
| 0 hours | >10⁵ | >10⁵ | >10⁵ | >10⁵ | >10⁵ | >10⁵ |
| 8 hours | >10⁵ | 0 | >10⁵ | >10⁵ | >10⁵ | >10⁵ |
| 24 hours | >10⁵ | 0 | >10⁵ | >10⁵ | >10⁵ | >10⁵ |

As the table shows, first, the silver sulfadiazine 1% cream has a great ability in inhibition of Enterococcus faecalis. Second, the traditional Shiunko has no ability in inhibition of Enterococcus faecalis. Third, the SSN, OSN, and MSN have no ability in inhibition of Enterococcus faecalis.

(IV) Proteus mirabilis

The calculating results of colony forming units of Proteus mirabilis are showed in table 6.

TABLE 6
the colony forming units of ATCC 7002 Proteus mirabilis

| | | silver | | | | |
|---|---|---|---|---|---|---|
| | control | sulfadiazine | traditional | | | |
| | group | 1% cream | Shiunko | SSN | OSN | MSN |
| 0 hours | >10⁵ | >10⁵ | >10⁵ | >10⁵ | >10⁵ | >10⁵ |
| 8 hours | >10⁵ | 0 | >10⁵ | >10⁵ | >10⁵ | >10⁵ |
| 24 hours | >10⁵ | 0 | >10⁵ | >10⁵ | >10⁵ | >10⁵ |

As the table shows, first, the silver sulfadiazine 1% cream has a great ability in inhibition of Proteus mirabilis. Second, the traditional Shiuriko has no ability in inhibition of Proteus mirabilis. Third, the SSN, OSN, and MSN have no ability in inhibition of Proteus mirabilis.

(IV) Acinetobacter baumannii

The calculating results of colony forming units of Acinetobacter baumannii are showed in table 7.

TABLE 7
the colony forming units of ATCC 19606 Acinetobacter baumannii

| | | silver | | | | |
|---|---|---|---|---|---|---|
| | control | sulfadiazine | traditional | | | |
| | group | 1% cream | Shiunko | SSN | OSN | MSN |
| 0 hours | >10⁵ | >10⁵ | >10⁵ | >10⁵ | >10⁵ | >10⁵ |
| 8 hours | >10⁵ | 0 | >10⁵ | >10⁵ | >10⁵ | >10⁵ |
| 24 hours | >10⁵ | 0 | >10⁵ | 120 | 140 | >10⁵ |

As the table shows, first, the silver sulfadiazine 1% cream has a great ability in inhibition of Acinetobacter baumannii. Second, the traditional Shiunko has no ability in inhibition of Acinetobacter baumannii. Third, the SSN, OSN, and MSN have a bad ability in inhibition of P Acinetobacter baumannii.

### Function Test III: Evaluation of Treating Effects in Burns and Scalds Injury

The experimental animals are twelve rabbits (male, New Zealand Rabbit), and the experiment is implemented in the Lab of Experimental Animal of Kaohsiung Medical University.

(I) Burn and Scald Model building

First, the experimental rabbits were washed with non-antibacterials soaps, and the dorsal fur was shaved off to expose the skin. Second, the rabbits are anaesthetized with ketamine 40 mg/kg i.m. Third, the twelve rabbits are divided into 6 groups (rabbits A, B are group 1; rabbits C, D are group 2; rabbits E, F are group 3; rabbits G, H are group 4; rabbits I, J are group 5; rabbits K, L are group 6,) and the predetermined injury regions of each rabbits are the same. Fourth, after heated in hot water having a temperature of 95° C. for 5 minutes, a iron column having a diameter of 2 cm is used to contact the exposed skin to produce 6 wounds respectively. After removing the iron and cooling the injury regions in room temperature for 10 minutes, the injury regions are treated with the different ointments or gels (the control group, the silver sulfadiazine 1% cream, the traditional Shiunko, the SSN gel, the OSN gel, and the MSN gel) clockwise in each group, and finally the transparent dressings (Tegaderm™) are put on each wounds respectively. Fifth, after recovering from anesthetization, the experimental rabbits are raised in normal condition. The ointments (or gels) and the dressings are replaced daily, and the wounds are recorded by photograph too. Sixth, from the time of 31 days after the injury, the ointments (or gels) and the dressings are replaced and the wounds are recorded every two days until all the rabbits heal. Seventh, after each treating, the rabbits are forced to wear special neck masks designed for preventing the rabbits licking the wounds. Eighth, the rabbits are sacrificed respectively at the time of 5, 10, 15, 20, 25 and 30 days after the injury, and the skin of the wound is took off by scissors and soaked in 10% Formalin. The tissue pathological variations of the skin are diagnosed under the direction of the pathological medicine specialist. Ninth, the 36 wounds of the other six rabbits are treated until healing, and the cutaneous wound regeneration and repair are observed and recorded.

### (II) Pathological Sections Preparing

The rabbits are sacrificed, and the wound tissues are sampled 3 cm×3 cm followed by soaking in 10% Fommalin. After dehydration, defatting and embedding, 3˜6 μm wound tissue sections are prepared by microtome and fixed on slides. After stained with Hematoxylin & Eosin, the wound tissue sections are observed by light microscope. Further, the tissue pathological variation are evaluated and scored depend on eight histomorphologic features of Hyperkeratosis, Epidermal hyperplasia, Hair follicles, Apocrine glands, Smooth muscles, Fibroplasia, Vascular proliferation and Collagen orientation respectively (Adam J. Singer et al., 2000).

(III) the Cutaneous Wound Regeneration and Repair Score Results

The cutaneous wound regeneration and repair are scored by the following standard:

5 points: the scabs have came off, and the wounds have healed, and the healing area is flat;
4 points: the scabs have came off, and the wounds have healed, and the healing area is raised;
3 points: the scabs have came off, and the wounds have not healed;
2 points: the scabs have formed over the wounds, and there is no exudate;
1 point: the scabs have formed over the wounds partially, and there is exudate sting.

The cutaneous wound regeneration and repair are scored at the time of 31 and 37 days after the injury, and the more points scored, the more well healing effects are evaluated. The cutaneous wound regeneration and repair scores are showed in tables 8 and 9.

TABLE 8

| | | silver | traditional | | | |
|---|---|---|---|---|---|---|
| | control | sulfadiazine 1% | Shiunko | | | |
| Day 31 | group | cream | ointment | SSN gel | OSN gel | MSN gel |
| rabbit A | 2 | 2 | 2 | 1 | 2 | 2 |
| rabbit B | 1 | 3 | 2 | 2 | 2 | 2 |
| rabbit H | 1 | 3 | 2 | 2 | 5 | 2 |
| rabbit J | 2 | 2 | 2 | 4 | 5 | 2 |
| rabbit K | 2 | 2 | 2 | 4 | 5 | 5 |
| rabbit L | 2 | 2 | 2 | 4 | 2 | 2 |
| total | 10 | 14 | 12 | 18 | 23 | 15 |
| mean | 1.67 ± 0.52 | 2.33 ± 0.52 | 2 ± 0 | 2.83 ± 1.33 | 3.50 ± 1.64 | 2.50 ± 1.22 |

TABLE 9

| | | silver | traditional | | | |
|---|---|---|---|---|---|---|
| | control | sulfadiazine 1% | Shiumko | | | |
| Day 37 | group | cream | ointment | SSN gel | OSN gel | MSN gel |
| rabbit A | 2 | 2 | 3 | 3 | 4 | 5 |
| rabbit B | 3 | 3 | 2 | 2 | 2 | 3 |
| rabbit H | 3 | 3 | 2 | 4 | 5 | 4 |
| rabbit J | 3 | 4 | 4 | 5 | 5 | 5 |
| rabbit K | 3 | 3 | 2 | 5 | 5 | 5 |
| rabbit L | 3 | 4 | 4 | 4 | 5 | 4 |
| total | 17 | 19 | 17 | 23 | 26 | 26 |
| mean | 2.83 ± 0.41 | 3.17 ± 0.75 | 2.83 ± 0.98 | 3.87 ± 1.17 | 4.33 ± 1.21 | 4.33 ± 0.81 |

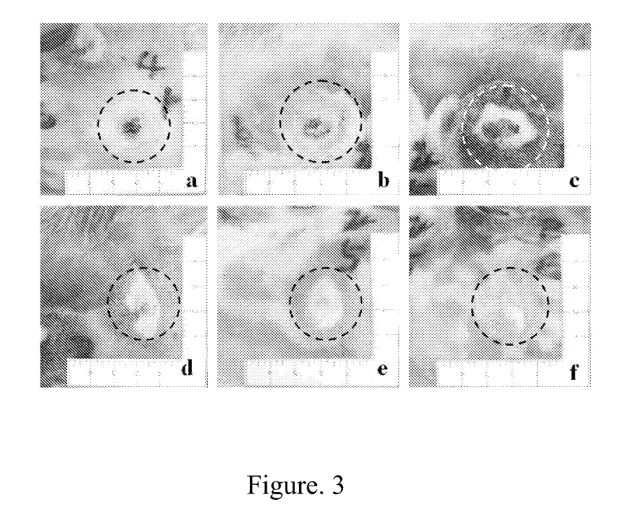

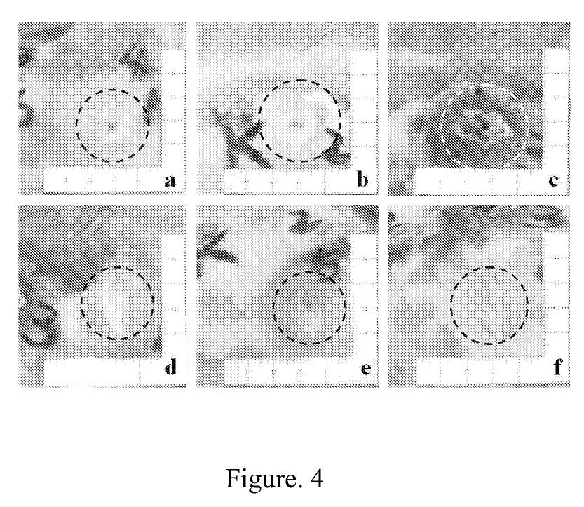

Please also refer to FIGS. 3 and 4, which are diagrams showing the wounds of the experimental rabbits at the time of 31 and 37 days after the burn injury and continuing treatment of no drugs(a), silver sulfadiazine 1% cream(b), traditional Shiunko(c), SSN(d), OSN(e), and MSN(f) respectively according to the embodiment of the present invention, no matter at 31 or 37 days, the scores of the wounds treated with SSN, OSN and MSN are better than other groups. Further, the cutaneous wound regenerating and repairing abilities of OSN and MSN are a bit better than SSM based on the scores of the time of 37 days, and OSM has a better ability in reducing the scar tissues.

On the other hand, the traditional Shiunko is prepared based on the wax, which has the disadvantages of hard-to-clean, being painful when replacing the dressings, and being disagreeable to the sight because of the residue having a red-purple color. However, all the disadvantages are overcomed by replacing the dressings with SSN, OSN or MSN gels.

(IV) the Tissue Pathological Variation

Please refer to table 11, which is a Histomorphologic Scale of the rabbit I at the time of day 5 after the injury.

TABLE 11
Histomorphologic Scale (Parameters and Scores) Day 5 (rabbt“I”)

| | Hyper- | | Epidermal | | | | Apocrine | | smooth | | | | vascular | | | | | | |
|---|---|---|---|---|---|---|---|---|---|---|---|---|---|---|---|---|---|---|---|
| | keratosis | | hyperplasia | | Hair follicles | | glands | | muscles | | Fibroplasia | | proliferation | | | | | | |
| | no | yes | no | yes | no | yes | no | yes | no | yes | no | yes | no | yes | collagen orientation | | | | |
| parameters | (1) | (0) | (1) | (0) | (1) | (0) | (1) | (0) | (1) | (0) | (1) | (0) | (1) | (0) | 3 | 2 | 1 | 0 | total |

| | | | | | | | | | | |
|---|---|---|---|---|---|---|---|---|---|---|
| control | 1 | 1 | 1 | 0 | 1 | 0 | 1 | 1 | 3: Normal | 6 |
| group | | | | | | | | | 2: Abnormal | |
| silver | 1 | 1 | 1 | 0 | 1 | 0 | 0 | 1 | collagen in | 5 |
| sulfadiazine | | | | | | | | | papillary | |
| 1% cream | | | | | | | | | dermis | |
| traditional | 1 | 1 | 1 | 0 | 0 | 0 | 0 | 1 | 1: Abnormal | 4 |
| Shiunko | | | | | | | | | collagen in | |
| ointment | | | | | | | | | upper | |
| SSN gel | 1 | 1 | 1 | 0 | 1 | 0 | 0 | 2 | reticular | 6 |
| OSN gel | 1 | 1 | 1 | 0 | 0 | 0 | 0 | 1 | dermis only | 4 |
| MSN gel | 1 | 1 | 1 | 0 | 1 | 1 | 1 | 2 | 0: Abnormal | 8 |
| | | | | | | | | | collagen in | |
| | | | | | | | | | upper and | |
| | | | | | | | | | lower half | |
| | | | | | | | | | of reticular | |
| | | | | | | | | | dermis | |

The scores are from 0 point of worst condition to 10 points of almost normal skin. The wound tissue sections are evaluated at the time of the days 5, 10, 15, 20, 25 and 30 after the injury, and the final scores are showed in table 10.

TABLE 10

| | Day 5 | Day 10 | Day 15 | Day 20 | Day 25 | Day 30 | total | mean |
|---|---|---|---|---|---|---|---|---|
| control | 6 | 3 | 4 | 1 | 4 | 4 | 22 | 3.67 ± 1.63 |
| group | | | | | | | | |
| silver | 5 | 1 | 2 | 3 | 5 | 4 | 20 | 3.33 ± 1.63 |
| sulfadiazine | | | | | | | | |
| 1% cream | | | | | | | | |
| traditional | 4 | 4 | 2 | 3 | 3 | 3 | 19 | 3.17 ± 0.75 |
| Shiunko | | | | | | | | |
| ointment | | | | | | | | |
| SSN gel | 6 | 2 | 4 | 4 | 6 | 5 | 27 | 4.5 ± 1.52 |
| OSN gel | 4 | 7 | 5 | 7 | 6 | 5 | 34 | 5.67 ± 1.21 |
| MSN gel | 8 | 6 | 5 | 7 | 7 | 6 | 39 | 6.5 ± 1.05 |

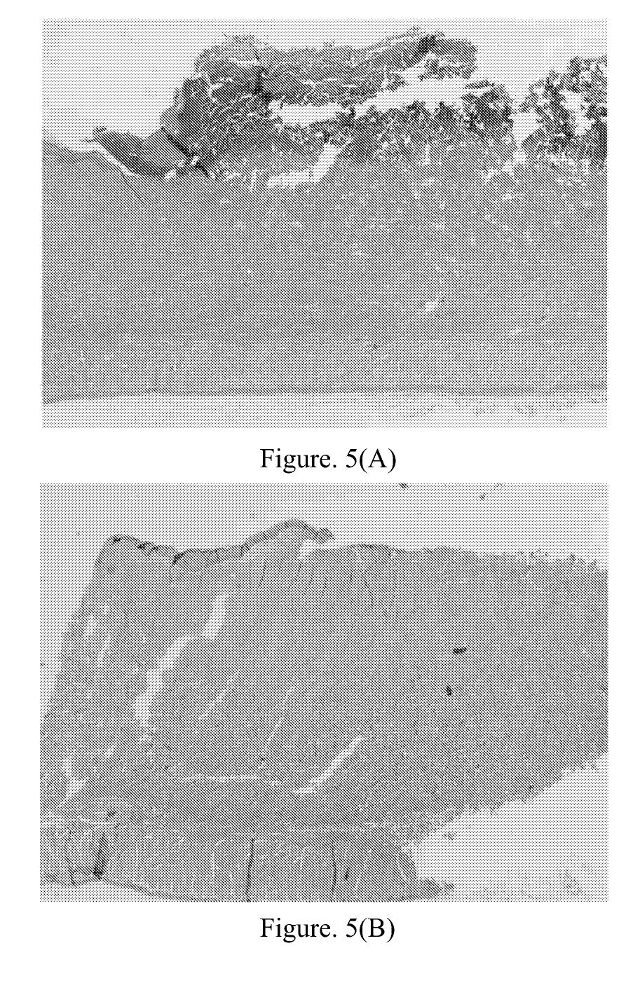

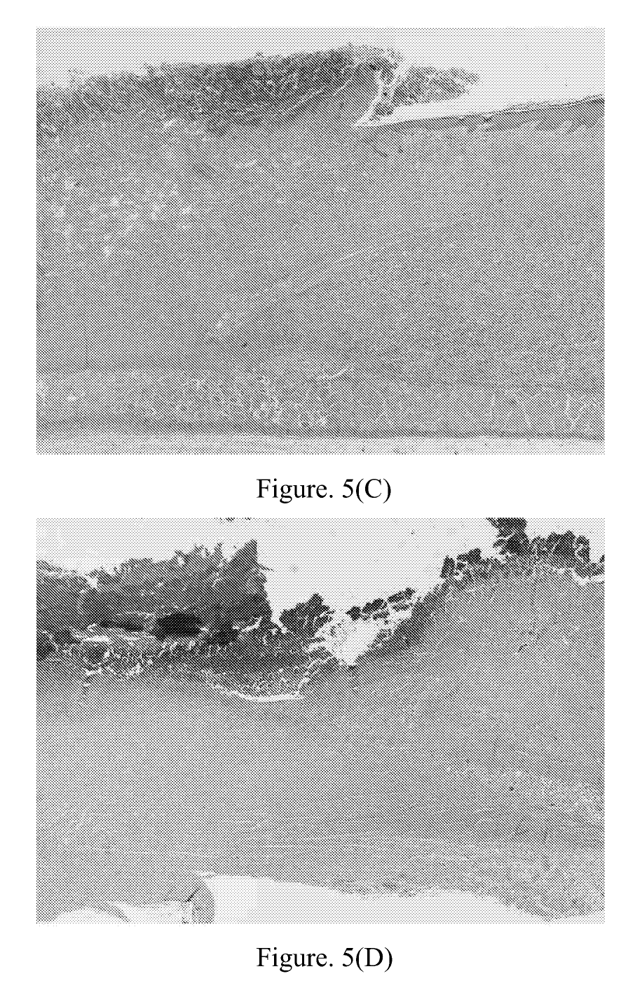

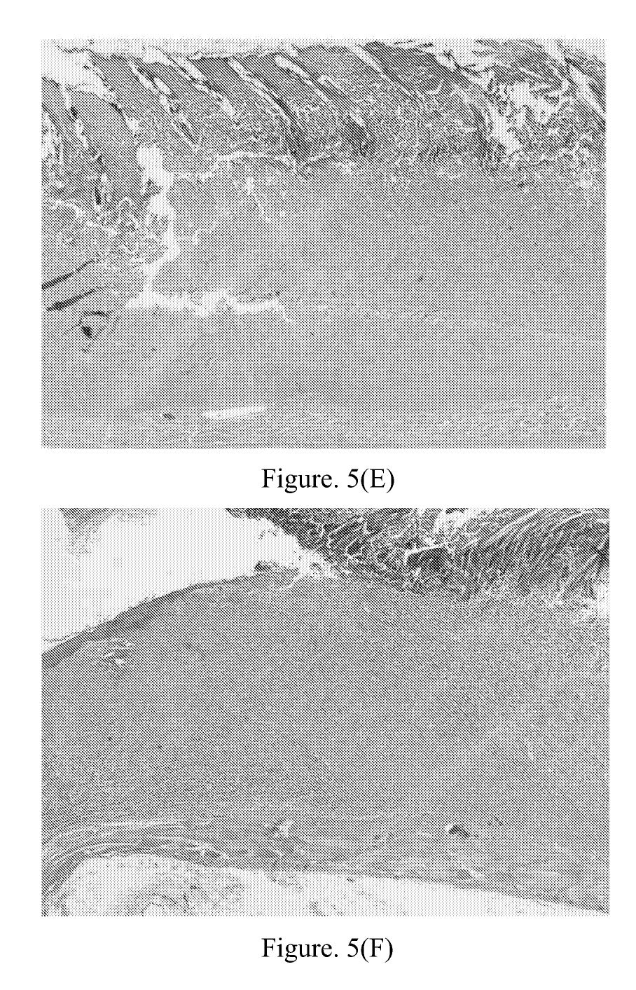

Please refer to table 10 and also FIGS. 5 (A)-(F), which are diagrams showing wound tissue sections in LM 20× after stained with Hematoxylin & Eosin at the time of 20 days after the burn injury and successive treatment of no drugs(a), silver sulfadiazine 1% cream(b), traditional Shiunko(c), SSN(d), OSN(e), and MSN(f) respectively according to the embodiment of the present invention. As the table 10 and the figures show, first, there are five dressings (control group, silver sulfadiazine 1% cream, traditional Shiunko ointment, SSN gel, MSN gel) having greater scores at the time of day 5 than those at the times of days 10, 15, 20, 25 and 30. Maybe it is because that the blood circulation of the wound tissues has not yet been cut off after burn injury, and hence the most wound tissues still have normal histomorphology. Second, based on the total scores, it is clear that the three different nanomicell gels have a better ability in wound repairing and healing than the control group, silver sulfadiazine 1% cream and traditional Shiunko. Further, the MSN gel has the best ability in wound repairing and healing than the OSN and the SSN gels, wherein the OSN gel is better than the SSN gel.

Based on the mention above, the SSN, the OSN and the MSN provided by the present invention, have a particle size below 100 nm even stored under the room temperature for 150 days, which shows the great stability. Further, the Shiunko nanomicell also shows the great ability in inhibition of Pseudomonas aeruginosa. As to the ability of wound repairing and healing, the Shiunko nanomicell obviously has the better ability than the traditional Shiunko prepared with wax and the silver sulfadiazine 1% cream. In addition, the Shiunko nanomicell is easy to be cleaned, and will not leave residues, which are all the benefits of the present invention.

From the evaluated results of the burn and scald model of rabbits, it shows that the OSN and the MSN gels have the better ability in wounds healing, however the OSN has a better ability in reducing scar. The evaluated results of wound tissue sections shows that the MSN has the greatest ability in healing than the OSN and SSN gels, and further the OSN is better than the SSN. However, no matter MSN, OSN, or SSN gel, is better than the control group, silver sulfadiazine 1% cream or traditional Shiunko. Accordingly, the Shiunko nanomicell gels provided by the present invention can be applied in burn and scald wounds to improve the healing and reduce the scar tissue, and further applied in relating skin diseases.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

### REFERENCES

1. Adam J. Singer, Henry C. Thode Jr., Steve A. McClain. Development of a histomorphologic scale to quantify cutaneous scars after burns. Acad Emerg Med. 2000, 7(10):1083-1088

What is claimed is:

1. A nanomicell for a skin, comprising:
an oil substance;
an extract of a Angelica Radix formed by extracting the Angelica Radix with the oil substance;
an extract of a Lithospermum Radix formed by extracting the Lithospermum Radix with the oil substance; and
a phospholipid layer, wherein the extract of the Angelica Radix and the extract of the Lithospermum Radix are packaged within the phospholipid layer to form a plurality of micells having a diameter of nano-level.

2. A nanomicell as claimed in claim 1, wherein the oil substance is one selected from a group consisting of a sesame oil, a mineral oil, and an olive oil.

3. A nanomicell as claimed in claim 1, wherein the phospholipids layer comprises a glycerin and a phospholipid.

4. A nanomicell as claimed in claim 1, wherein the micell further comprises an agueous layer outside the phospholipids layer.

5. A nanomicell as claimed in claim 1, wherein the micell has a diameter lesser than 100 nanometers.

6. A nanomicell as claimed in claim 1, wherein the nanomicell is used for a treatment for one selected from a group consisting of wound injury, burn injury, dry skin, fissure, frostbite, ulcer, and proliferate skin diseases.

7. A nanomicell gel for a skin treatment, comprising:
an excipient having a plurality of micells distributed therein, wherein each of the plurality of micells comprises
an oil extract of Angelica Radix;
an oil extract of Lithospermum Radix; and
a phospholipid layer, wrapping therewithin the oil extract of Angelica Radix and the oil extract of Lithospermum Radix.

8. A nanomicell gel as claimed in claim 7, wherein the phospholipid layer comprises a glycerin and a phospholipid

9. A nanomicell gel as claimed in claim 7, wherein each of the plurality of micells further comprises a aqueous layer outside the phospholipid layer.

10. A nanomicell gel as claimed in claim 7, wherein the excipient is selected from one of a gel and a hydrophilic ointment.

11. A nanomicell gel as claimed in claim 7, wherein each of the plurality of micells has a diameter lesser than 100 nanometers.

12. A nanomicell gel as claimed in claim 7, wherein the nanomicell gel is used for a treatment for one selected from a group consisting of wound injury, burn injury, dry skin, fissure, frostbite, ulcer, and proliferate skin diseases.

13. A method of forming a nanomicell for a skin, comprising steps of:
(a) extracting an Angelica Radix and a Lithospermum Radix with an oil substance to obtain an extract;
(b) adding a phospholipid and a water into the extract;
(c) stirring the extract in a relatively high speed and relatively high pressure condition to homogenize the extract; and
(d) filtrating the extract.

14. A method as claimed in claim 13, further comprising a step of soaking the Angelica Radix in the oil substance for 24 hours before the step of extracting an Angelica Radix and a Lithospermum Radix.

15. A method as claimed in claim 13, further comprising a step of heating the oil substance to a temperature between 130° C. and 140° C. to extract the Angelica Radix and the Lithospermum Radix.

16. A method as claimed in claim 13, wherein the step (d) is carried out by a filter having a pore size below 0.1 μm

17. A method as claimed in claim 13, wherein the oil substance is one selected from a group consisted of a sesame oil, a mineral oil, and an olive oil.

18. A method as claimed in claim 13, further comprising a step of add a excipient to the extract after the step of filtrating the extract.

19. A method as claimed in claim 18, wherein the excipient is one selected from a gel and a hydrophilic ointment.
